(19) United States
(12) Patent Application Publication
(10) Pub. No.: US 2020/0261111 A1
(43) Pub. Date: Aug. 20, 2020

(54) ATHERECTOMY DEVICE HAVING AN ULTRASONIC CATHETER ASSEMBLY

(71) Applicant: C. R. Bard, Inc., Franklin Lakes, NJ (US)

(72) Inventor: Scott L. Randall, Mesa, AZ (US)

(21) Appl. No.: 16/278,466

(22) Filed: Feb. 18, 2019

(51) Int. Cl. A61B 17/3207; A61B 17/22

(52) U.S. Cl. CPC A61B 17/320758; A61B 2017/22001; A61B 2017/320775; A61B 17/22004

(57) ABSTRACT

An atherectomy device includes a handpiece having an ultrasonic transducer, and an ultrasonic catheter assembly detachably attached to the handpiece. The ultrasonic catheter assembly includes a sheath having a proximal end, a distal end, and an elongate lumen. A core wire is located in the elongate lumen. The core wire has a distal tip and a distal portion proximal to the distal tip. The distal portion has an abrasive exterior surface portion. The abrasive exterior surface portion defines a plurality of cutting protrusions, wherein each of the plurality of cutting protrusions extends away from the core wire in a distal direction at an acute angle with respect to a longitudinal external surface of the core wire.

### CROSS-REFERENCE TO RELATED APPLICATIONS

None.

### TECHNICAL FIELD

The present invention relates to medical devices for removing lesions from a blood vessel, and, more particularly, to an atherectomy device.

### BACKGROUND ART

Surgical procedures, such as angioplasty or atherectomy, can be used to restore patency and blood flow that was lost due to one or more intravascular lesions. One type of atherectomy device has a smooth atherectomy wire that works well on intravascular lesions formed by calcific material, but does not work well in breaking up intravascular lesions that include fibrous material. Other approaches may use a spinning burr on the distal end of a rotating drive wire.

What is needed in the art is an improved atherectomy device that is capable of breaking up both calcified and fibrous lesions in a blood vessel.

### SUMMARY OF INVENTION

The present invention provides an improved atherectomy device, and ultrasonic catheter assembly, that is capable of breaking up both calcified and fibrous lesions in a blood vessel.

The invention in one form is directed to an atherectomy device that includes a handpiece having an ultrasonic transducer, and an ultrasonic catheter assembly detachably attached to the handpiece. The ultrasonic catheter assembly includes a sheath having a proximal end, a distal end, and an elongate lumen. A core wire is located in the elongate lumen. The core wire has a distal tip and a distal portion proximal to the distal tip. The distal portion has an abrasive exterior surface portion. The abrasive exterior surface portion defines a plurality of cutting protrusions, wherein each of the plurality of cutting protrusions extends away from the core wire in a distal direction at an acute angle with respect to a longitudinal external surface of the core wire.

The invention in another form is directed to an ultrasonic catheter assembly that includes a sheath having a proximal end, a distal end, and an elongate lumen. A core wire is located in the elongate lumen. The core wire has a distal tip and a distal portion proximal to the distal tip. The distal portion has an abrasive exterior surface portion. The abrasive exterior surface portion defines a plurality of cutting protrusions, wherein each of the plurality of cutting protrusions extends away from the core wire in a distal direction at an acute angle with respect to a longitudinal external surface of the core wire.

An advantage of the present invention is that an ultrasonic system having the core wire with the abrasive exterior surface portion as described herein is capable of breaking up both calcified and fibrous lesions in a blood vessel. In addition, advantageously, having the plurality of cutting protrusions that extends away from the core wire in a distal direction at an acute angle with respect to a longitudinal external surface of the core wire may effect dislodging, cutting, and/or morcellating of the lesions in the blood vessel.

### BRIEF DESCRIPTION OF DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematic illustrating an ultrasonic system for performing an atherectomy procedure, in accordance with an embodiment of the present invention.

FIG. 2 is an enlarged side view of a distal portion of a core wire of the ultrasonic system of FIG. 1, having an abrasive exterior surface portion that defines a plurality of cutting protrusions.

FIG. 3 is a further enlargement of a cutting protrusion of the abrasive exterior surface portion of FIG. 2.

FIG. 4A is a side view of an alternative arrangement of an abrasive exterior surface portion having a plurality of circumferentially separated longitudinal bands.

FIG. 4B is a section view of the alternative arrangement of FIG. 4A taken along line 4B-4B.

FIG. 5 is a side view of an alternative arrangement of an abrasive exterior surface portion having a plurality of longitudinally separated annular bands.

FIG. 6 is a side view of an alternative arrangement of an abrasive exterior surface portion formed as a spiral pattern that spirals around the circumference of the core wire.

Corresponding reference characters indicate corresponding parts throughout the several views. The exemplifications set out herein illustrate embodiments of the invention, and such exemplifications are not to be construed as limiting the scope of the invention in any manner.

### DESCRIPTION OF EMBODIMENTS

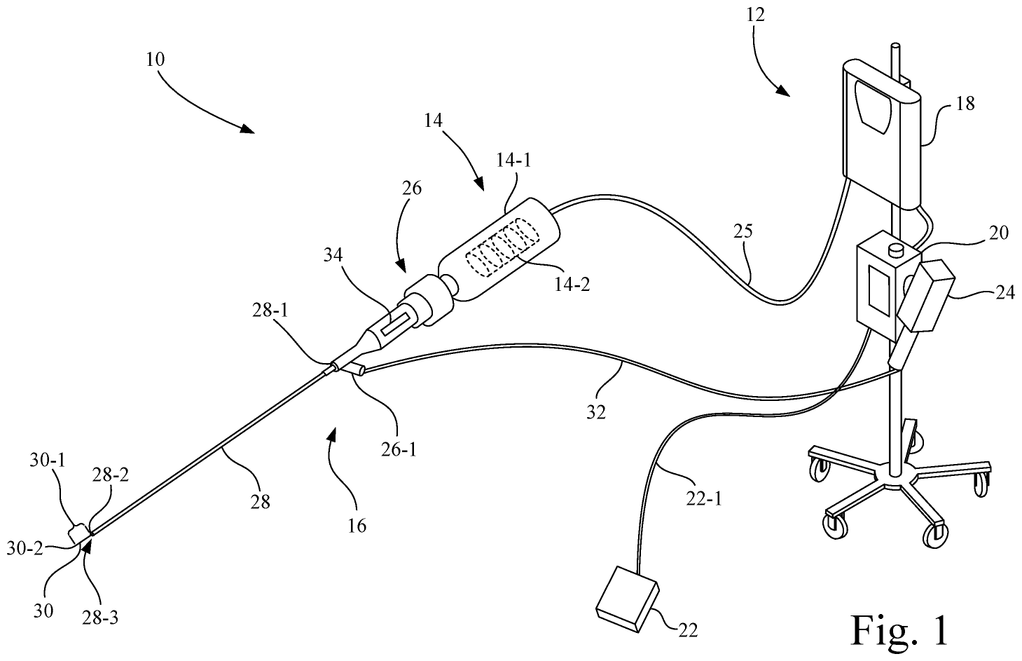

Referring now to the drawings, and more particularly to FIG. 1, there is shown an ultrasonic system 10 for performing an atherectomy procedure, in accordance with an embodiment of the present invention.

Ultrasonic system 10 may include a console 12, a handpiece 14, and an ultrasonic catheter assembly 16. Ultrasonic catheter assembly 16 is detachably attached to handpiece 14, and together form an atherectomy device.

Console 12 includes an ultrasonic signal generator 18 and a controller 20, and may optionally include a foot switch 22 and/or a saline injector 24. Controller 20 includes processing circuitry, interface circuitry, and electronic memory. In the present embodiment, controller 20 is electrically connected and communicatively coupled to each of ultrasonic signal generator 18, foot switch 22, and saline injector 24.

Ultrasonic signal generator 18 is configured to convert an alternating electric current, e.g., from a wall outlet, into a high-frequency current. Foot switch 22 has an electrical cable 22-1 that is connected to controller 20 and provides operational signals to controller 20 to activate and deactivate the components, e.g., ultrasonic signal generator 18 and saline injector 24, of ultrasonic system 10.

Saline injector 24 delivers sterile saline that may be used to irrigate an anatomical area undergoing an intravascular lesion-modification procedure and/or for cooling motional components of the ultrasonic catheter assembly 16.

In the present embodiment, handpiece 14 includes a housing body 14-1 and an ultrasonic transducer 14-2 mounted internally to housing body 14-1. Housing body 14-1 has an outer shape and size to facilitate being grasped by an operator during an atherectomy procedure. Ultrasonic transducer 14-2 may be, for example, a piezoelectric-type transducer. Ultrasonic transducer 14-2 of handpiece 14 is electrically connected to ultrasonic signal generator 18 by electrical cable 25, and is configured to convert the high-frequency current produced by ultrasonic signal generator 18 into vibrational energy, which may be in a frequency range, for example, of 18 kHz to 40 kHz, and may be a fixed frequency or a variable frequency.

In the present embodiment, ultrasonic catheter assembly 16 includes a housing 26, a sheath 28, and a core wire 30.

Housing 26 includes a Y-connector 26-1 that provides access to an irrigation lumen (not shown) of sheath 28. Y-connector 26-1 is connected, e.g., by a flexible hose 32, to saline injector 24. In the present embodiment, housing 26 further includes a retraction-extension mechanism 34, such as a slide, that is connected to sheath 28.

Sheath 28 is an elongate flexible tube, such a polymer tube. Sheath 28 includes a proximal end 28-1, a distal end 28-2, and a sheath lumen 28-3. In the present embodiment, sheath lumen 28-3 is an elongate lumen that longitudinally extends within sheath 28 from proximal end 28-1 to distal end 28-2, and may be formed as a central lumen, relative to the diameter, of sheath 28. Proximal end 28-1 of sheath 28 is connected to housing 26, and more particularly in the present embodiment, to retraction-extension mechanism 34.

Core wire 30 is located in, and longitudinally extends within, sheath lumen 28-3. Core wire 30 is an elongate flexible metal wire, e.g., nitinol, which is slidably received in sheath lumen 28-3 of sheath 28. In the present embodiment, core wire 30 may have a length greater than 60 centimeters (cm), and in some embodiments, a length of 100 to 200 cm. Core wire 30 is operably connected to ultrasonic transducer 130 to receive the vibrational energy from ultrasonic transducer 130 so as to produce a vibrational motion of core wire 30. The vibrational motion of core wire 30 may be longitudinal and/or transverse. Core wire 30 may be activated to vibrate, for example, by operation of foot switch 22.

Retraction-extension mechanism 34 of housing 26 is configured to retract sheath 28 from a first, fully extended position of sheath 28, in which core wire 30 is wholly disposed within the sheath lumen 28-3 of sheath 28, to a fully retracted position of sheath 28, in which a distal portion 30-1 of core wire 30, e.g., 5 cm to 8 cm, extends distally from distal end 28-2 of sheath 28. Alternatively, however, it is contemplated that core wire 30 may be slidably moved relative to sheath 28 to fully extend core wire 30 to expose distal portion 30-1 of core wire 30 distally from distal end 28-2 of sheath 28, and to fully retract core wire 30 to cover distal portion 30-1 of core wire 30 with sheath 28.

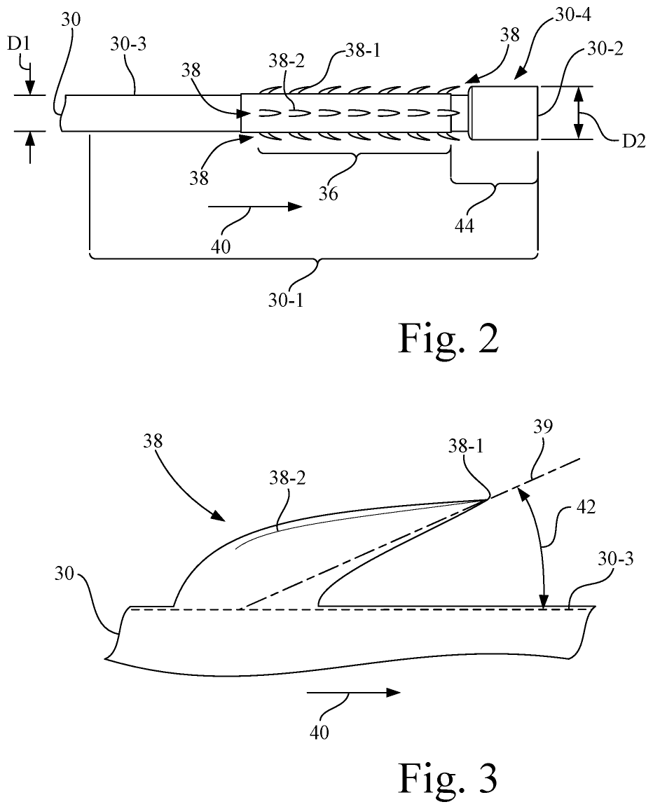

Referring also to FIG. 2, core wire 30 has a distal tip 30-2, with distal portion 30-1 being proximal to, and distally terminating, at distal tip 30-2. Core wire 30 has a longitudinal external surface 30-3. Distal tip 30-2 of core wire 30 may be, for example, a blunt tip.

Distal portion 30-1 has an abrasive exterior surface portion 36 formed on, or attached to, longitudinal external surface 30-3. In the present embodiment, abrasive exterior surface portion 36 extends annularly around an entire circumference of core wire 30. Abrasive exterior surface portion 36 defines a plurality of cutting protrusions 38.

Referring also to FIG. 3, each of the plurality of cutting protrusions 38 may be in the form of an arcuate barb having an axis 39 that that is oriented to extend through a centroid of the base of the barb to the tip of the barb, such that each of the plurality of cutting protrusions 38 is oriented to extend away from core wire 30, e.g., in a distal direction 40, at an acute angle 42 with respect to the longitudinal external surface 30-3 of core wire 30. In other words, each of the plurality of cutting protrusions 38, i.e., each barb, is oriented to extend in distal direction 40 and to diverge at acute angle 42 away from the longitudinal external surface 30-3 of core wire 30. It is contemplated that the acute angle 42 for each of the plurality of cutting protrusions 38 need not be, but may be, the same acute angular value.

In the present embodiment, each barb of the plurality of cutting protrusions 38 distally tapers to form a sharp distal tip 38-1. Also, each barb may include one or more of an exterior cutting edge 38-2, e.g., one or more side cutting edges. In some embodiments, the exterior cutting edge 38-2 may terminate at the sharp distal tip 38-1.

In the present embodiment, abrasive exterior surface portion 36 is located on core wire 30 a distance 44 proximal to distal tip 30-2 of core wire 30. Distance 44 may be, for example, in a range of 0.2 to 2.0 cm.

Core wire 30 has an enlarged diameter portion 30-4 that extends proximally from distal tip 30-2 and terminates distal to abrasive exterior surface portion 36. Accordingly, in the present embodiment, core wire 30 has a first diameter D1 proximal to abrasive exterior surface portion 36, and a second diameter D2 of enlarged diameter portion 30-4 that is distal to abrasive exterior surface portion 36, with second diameter D2 being larger than first diameter D1. First diameter D1 of core wire 30 may be, for example, 0.28 millimeters (mm)±5 percent, and second diameter D2 of core wire 30 may be, for example, 0.76 mm±5 percent. In the present embodiment, enlarged diameter portion 30-4 of core wire 30 having second diameter D2 is sized to be received in a snug sliding fit in sheath lumen 28-3 of sheath 28.

It is contemplated that abrasive exterior surface portion 36 on longitudinal external surface 30-3 in distal portion 30-1 of core wire 30 may be formed using any of a multitude of techniques, such as by punching or cutting (e.g., plasma or laser) the longitudinal external surface 30-3 in distal portion 30-1 of core wire 30 to form the plurality of cutting protrusions 38, or by applying an abrasive coating to the longitudinal external surface 30-3 in distal portion 30-1 of core wire 30 to form the plurality of cutting protrusions 38.

For example, as an abrasive coating, the coating material, e.g., carbide or metal particulate, may be electrostatically applied to longitudinal external surface 30-3 in distal portion 30-1 of core wire 30, and permanently adhered to longitudinal external surface 30-3 by adhesive or weld to form the plurality of cutting protrusions 38.

In the present embodiment, retraction-extension mechanism 34 of housing 26 is configured to retract sheath 28 from the first, fully extended position of sheath 28, in which abrasive exterior surface portion 36 on longitudinal external surface 30-3 in distal portion 30-1 of core wire 30 is wholly disposed within the sheath lumen 28-3 of sheath 28, to the fully retracted position of sheath 28, in which abrasive exterior surface portion 36 on longitudinal external surface 30-3 in distal portion 30-1 of core wire 30 extends distally from distal end 28-2 of sheath 28. Alternatively, however, it is contemplated that core wire 30 may be slidably moved relative to sheath 28 to fully extend core wire 30 to expose the abrasive exterior surface portion 36 on longitudinal external surface 30-3 in distal portion 30-1 of core wire 30 distally from distal end 28-2 of sheath 28, and to fully retract core wire 30 to cover the abrasive exterior surface portion 36 on longitudinal external surface 30-3 in distal portion 30-1 of core wire 30 with sheath 28.

In operation, with abrasive exterior surface portion 36 exposed distally from distal end 28-2 of sheath 28, ultrasonic vibrations generated by ultrasonic transducer 14-2 are transmitted via core wire 30 to abrasive exterior surface portion 36, wherein the plurality of cutting protrusions 38 of abrasive exterior surface portion 36 are vibrated in a longitudinal and/or transverse motion, so as to engage and remove calcified and/or fibrous lesions in a blood vessel during an atherectomy procedure.

While in the present embodiment, abrasive exterior surface portion 36 extends annularly around an entire circumference of core wire 30, it is contemplated that other arrangements are possible.

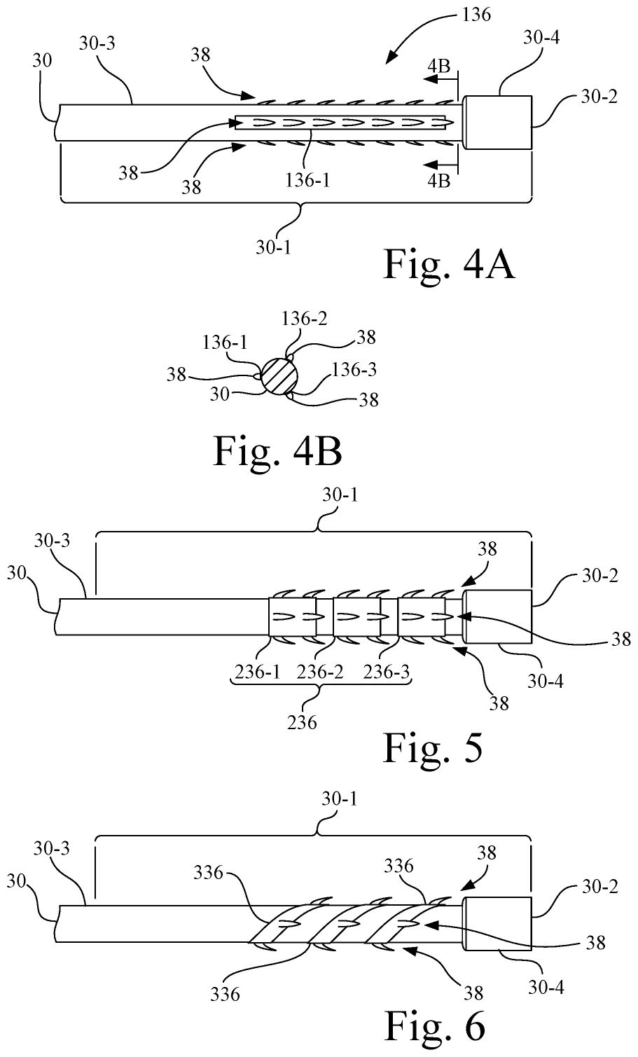

For example, FIGS. 4A and 4B show an alternative arrangement of an abrasive exterior surface portion 136 having a plurality of circumferentially separated longitudinal bands, and in the present embodiment, includes three circumferentially separated longitudinal bands individually identified as bands 136-1, 136-2, and 136-3, which may be spaced at 120 degree increments around the circumference of core wire 30. Each of the plurality of circumferentially separated longitudinal bands 136-1, 136-2, and 136-3 includes a respective plurality of cutting protrusions 38.

FIG. 5 shows an alternative arrangement of an abrasive exterior surface portion 236 having a plurality of longitudinally separated annular bands, and in the present embodiment, includes three longitudinally separated annular bands individually identified as longitudinally separated annular bands 236-1, 236-2, and 236-3. Each of the plurality of longitudinally separated annular bands 236-1, 236-2, and 236-3 includes a respective plurality of cutting protrusions 38.

FIG. 6 shows an alternative arrangement of an abrasive exterior surface portion 336 formed as a spiral pattern 336-1 that spirals around the circumference of core wire 30. The spiral pattern 336-1 of abrasive exterior surface portion 336 includes a respective plurality of cutting protrusions 38.

The various arrangements of abrasive exterior surface portions 36, 136, 236, and 336 are provided as examples of patterns having the plurality of cutting protrusions 38, and it is contemplated that other patterns, variations, and combinations thereof having the plurality of cutting protrusions 38 are possible and fall within the scope of this invention.

For definitional purposes and as used herein, “connected” or “attached” includes direct or indirect connection.

While this invention has been described with respect to several embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An atherectomy device, comprising:
a handpiece having an ultrasonic transducer; and
an ultrasonic catheter assembly detachably attached to the handpiece, the ultrasonic catheter assembly including:
a sheath having a proximal end, a distal end, and an elongate lumen; and
a core wire located in the elongate lumen, the core wire having a distal tip and a distal portion proximal to the distal tip, the distal portion having an abrasive exterior surface portion, the abrasive exterior surface portion defining a plurality of cutting protrusions, wherein each of the plurality of cutting protrusions extends away from the core wire in a distal direction at an acute angle with respect to a longitudinal external surface of the core wire.

2. The atherectomy device of claim 1, wherein the distal portion extends distally from the distal end of the sheath.

3. The atherectomy device of claim 1, wherein the abrasive exterior surface portion is positioned a distance proximal to the distal tip of the core wire.

4. The atherectomy device of claim 3, wherein the core wire has a first diameter proximal to the abrasive exterior surface portion, and a second diameter distal to the abrasive exterior surface portion, the second diameter being larger than the first diameter.

5. The atherectomy device of claim 1, wherein the abrasive exterior surface portion extends annularly around an entire circumference of the core wire.

6. The atherectomy device of claim 1, wherein the abrasive exterior surface portion is formed as a plurality of bands.

7. The atherectomy device of claim 6, wherein the plurality of bands is a plurality of circumferentially separated longitudinal bands, with each band of the plurality of circumferentially separated longitudinal bands including a respective plurality of the cutting protrusions.

8. The atherectomy device of claim 6, wherein the plurality of bands is a plurality of longitudinally separated annular bands, with each band of the plurality of longitudinally separated annular bands including a respective plurality of the cutting protrusions.

9. The atherectomy device of claim 1, wherein the abrasive exterior surface portion is formed as a spiral pattern that spirals around a circumference of the core wire.

10. The atherectomy device of claim 1, wherein the abrasive exterior surface portion having the plurality of cutting protrusions is formed as one or more of a plurality of circumferentially separated longitudinal bands, a plurality of circumferentially separated longitudinal bands, and a spiral pattern.

11. An ultrasonic catheter assembly, comprising:
a housing;
a sheath connected to the housing, the sheath having a proximal end, a distal end, and an elongate lumen; and
a core wire located in the elongate lumen, the core wire having a distal tip and a distal portion proximal to the distal tip, the distal portion having an abrasive exterior surface portion, the abrasive exterior surface portion defining a plurality of cutting protrusions, wherein each of the plurality of cutting protrusions extends away from the core wire in a distal direction at an acute angle with respect to a longitudinal external surface of the core wire.

12. The ultrasonic catheter assembly of claim 11, wherein the distal portion extends distally from the distal end of the sheath.

13. The ultrasonic catheter assembly of claim 11, wherein the abrasive exterior surface portion is positioned a distance proximal to the distal tip of the core wire.

14. The ultrasonic catheter assembly of claim 13, wherein the core wire has a first diameter proximal to the abrasive exterior surface portion, and a second diameter distal to the abrasive exterior surface portion, the second diameter being larger than the first diameter.

15. The ultrasonic catheter assembly of claim 11, wherein the abrasive exterior surface portion extends annularly around an entire circumference of the core wire.

16. The ultrasonic catheter assembly of claim 11, wherein the abrasive exterior surface portion is formed as a plurality of bands.

17. The ultrasonic catheter assembly of claim 16, wherein the plurality of bands is a plurality of circumferentially separated longitudinal bands, with each band of the plurality of circumferentially separated longitudinal bands including a respective plurality of the cutting protrusions.

18. The ultrasonic catheter assembly of claim 16, wherein the plurality of bands is a plurality of longitudinally separated annular bands, with each band of the plurality of longitudinally separated annular bands including a respective plurality of the cutting protrusions.

19. The ultrasonic catheter assembly of claim 11, wherein the abrasive exterior surface portion is formed as a spiral pattern that spirals around a circumference of the core wire.

20. The ultrasonic catheter assembly of claim 11, wherein the abrasive exterior surface portion having the plurality of cutting protrusions is formed as one or more of a plurality of circumferentially separated longitudinal bands, a plurality of circumferentially separated longitudinal bands, and a spiral pattern.
